(19) United States
(12) Patent Application Publication
(10) Pub. No.: US 2026/0335053 A1
(43) Pub. Date: Oct. 29, 2026

(54) PACKAGING DEVICE FOR A DENTAL PROSTHESIS

(71) Applicant: EUROTEKNIKA, Sallanches (FR)

(72) Inventors: Rudy Lo Savio Vogel, Domancy (FR); Cédric Lancieux, Megève (FR)

(73) Assignee: EUROTEKNIKA, Sallanches (FR)

(21) Appl. No.: 19/110,256

(22) PCT Filed: Sep. 11, 2023
(86) PCT No.: PCT/EP2023/074921

§ 371 Date: Mar. 10, 2025

(30) Foreign Application Priority Data
Sep. 12, 2022 (FR) FR2209112

(51) Int. Cl. A61C 8/00; A61B 17/86; A61B 50/30; B65B 5/04

(52) U.S. Cl. CPC A61C 8/0087; A61B 17/865; A61B 50/30; B65B 5/04; A61B 2050/3015

(57) ABSTRACT

The packaging device (1) for a dental prosthesis (P) has a base (2) with a support (3) intended to hold the dental prosthesis, an envelope (4) formed of a first part (5) and a second part (6), and at least one arm (7) connecting the first part of the envelope to the base. The packaging device is able to transition from a closed configuration to an open configuration, the base having at least one pivot (12) for the at least one arm, a force applied to the at least one arm causing the packaging device to transition from its closed configuration to its open configuration.

### TECHNICAL FIELD OF THE INVENTION

The invention relates to the field of packaging devices for a dental prosthesis, in particular for a dental implant. The invention also relates to a method for manufacturing such a packaging device, a method for packaging a dental prosthesis in such a packaging device, and a method for unpackaging a dental prosthesis packaged in such a packaging device.

### BACKGROUND ART

Dental prostheses, such as dental implants, are intended to be integrated into the mouth of a patient in order to restore the latter's teeth. Dental prostheses must be of impeccable quality and cleanliness at the time they are integrated into the mouth. Between their manufacture and their use by a dentist, dental prostheses are generally packaged in individual packaging devices. The packaging devices must be able to protect the dental prostheses from any risk of damage or contamination between their manufacture and their use. The publications US2007295620A1 and EP0963738A1 disclose examples of packaging devices. The known packaging devices are generally too complex and/or impractical to use, in particular in the context of dental care where the dentist has to be able to unpackage the dental prosthesis efficiently and quickly.

### SUMMARY OF THE INVENTION

The aim of the invention is to provide a packaging device for a dental prosthesis that overcomes the above disadvantages and improves the packaging devices known from the prior art.

More specifically, a first subject of the invention is a packaging device that is simple to manufacture and to use.

The invention relates to a packaging device for a dental prosthesis, comprising:
a base comprising a support intended to hold the dental prosthesis,
an envelope formed of a first part and of a second part, and
at least one arm comprising a first end and a second end, the first end being connected to the base, the second end being connected to the first part of the envelope,
the packaging device being able to transition from a closed configuration to an open configuration, the first part and the second part of the envelope being brought close together to enclose the dental prosthesis when the packaging device is in the closed configuration, the first part and the second part of the envelope being spaced apart from each other to provide access to the dental prosthesis when the packaging device is in the open configuration,
the base comprising at least one pivot means for the at least one arm, a force applied to the at least one arm, between its first end and the at least one pivot means, causing the packaging device to transition from its closed configuration to its open configuration.

The packaging device can comprise a first arm and a second arm, each arm comprising a first end and a second end, the first end of each arm being connected to the base, the second end of the first arm being connected to the first part of the envelope, the second end of the second arm being connected to the second part of the envelope, the base comprising a first pivot means for the first arm and a second pivot means for the second arm, a force applied to the first arm, between its first end and the first pivot means, and a force applied to the second arm, between its first end and the second pivot means, causing the packaging device to transition from its closed configuration to its open configuration.

The packaging device can be in one-piece and obtained by plastic injection.

The base can comprise at least one retention means cooperating with the at least one arm in order to hold the packaging device in a closed configuration.

The retention means can comprise at least one deformable tab, the tab being intended to deform in order to allow the movement of the at least one arm when the packaging device transitions from an original configuration at the end of its manufacture to its closed configuration.

The packaging device can comprise a shape memory that tends to cause it to transition to its closed configuration.

The base can comprise a generally elongate shape, the first end of the at least one arm being connected to a first end of the base, the support being arranged at a second end of the base, the second end of the base being opposite the first end of the base.

The base can further comprise at least one housing for a fastening screw able to cooperate with the dental prosthesis. The first part and the second part of the envelope can be two half-shells of the envelope. The first part and/or the second part of the envelope can be made of transparent plastic material and can have graduations in relief, which are configured for estimating a size of the dental prosthesis.

The packaging device can comprise a fastening clip arranged between the two parts of the envelope, for maintaining the packaging device in a closed configuration.

The invention also relates to a kit comprising a packaging device as defined above and a dental prosthesis fastened to the support of the base.

The invention also relates to a method for manufacturing a packaging device as defined above, the packaging device being manufactured by plastic injection in an original configuration in which the at least one arm has the shape of an arc of a circle and in which the at least one arm is spaced apart from the at least one pivot means.

The invention also relates to a method for packaging a dental prosthesis in a packaging device as defined above, the packaging method comprising:
manufacturing the packaging device by plastic injection, then
fastening a dental prosthesis to the support of the base, then
bringing the first part of the envelope and the second part of the envelope together around the dental prosthesis.

The invention also relates to a method for unpackaging a dental prosthesis packaged in a packaging device as defined above, comprising pressing against the at least one arm, between its first end and the at least one pivot means, in order to cause the at least one arm to bend in the direction of the base, the bending of the at least one arm causing the packaging device to open.

### BRIEF DESCRIPTION OF THE DRAWINGS

These subjects, features and advantages of the present invention will be set out in detail in the following description of a particular embodiment, given by way of non-limiting example with reference to the appended figures, in which:

FIG. 1 is a perspective view of a packaging device according to one embodiment of the invention, the packaging device being in an original configuration.

FIG. 2 is a perspective view of the packaging device in a closed configuration.

FIG. 3 is a profile view of the packaging device in its original configuration.

FIG. 4 is a profile view of the packaging device in a closed configuration.

FIG. 5 is a schematic profile view of the kinematics of closure of the packaging device around a dental prosthesis.

FIG. 6 is a profile view of the packaging device, shown as transparent, in a closed configuration, the dental prosthesis being held in a support of the packaging device.

FIG. 7 is a schematic profile view of the packaging device during its opening.

FIG. 8 is a schematic profile view of the packaging device in an open configuration.

FIG. 9 is a perspective and exploded view of a kit comprising the packaging device, the dental prosthesis, and two fastening screws able to cooperate with the dental prosthesis.

FIG. 10 is a profile view of a first variant embodiment of a packaging device.

FIG. 11 is a profile view of a second variant embodiment of a packaging device, the packaging device being in its original configuration.

FIG. 12 is a profile view of the second variant embodiment of the packaging device, the packaging device being in a closed configuration.

### DETAILED DESCRIPTION OF PARTICULAR EMBODIMENTS

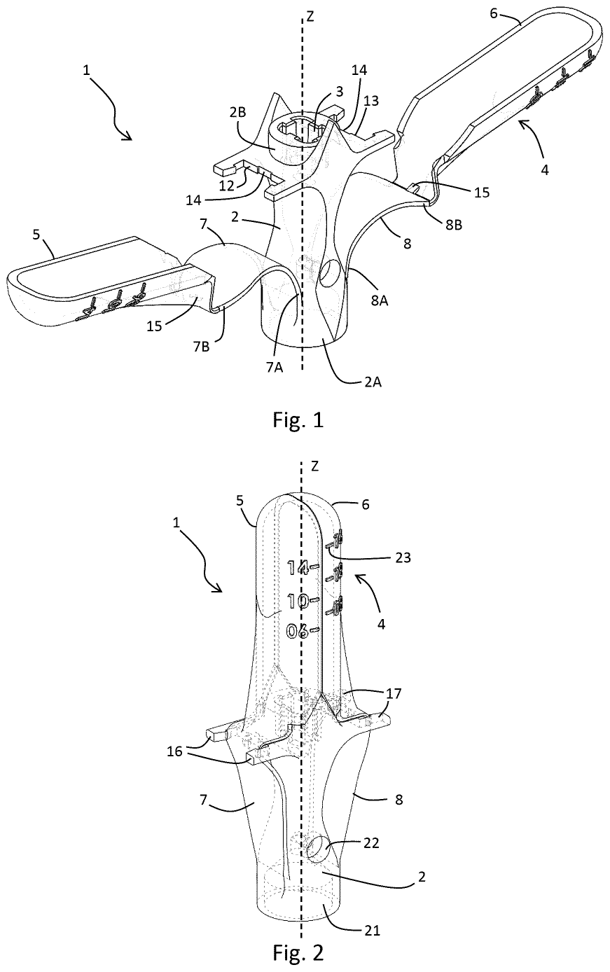

FIG. 1 illustrates a packaging device 1 for a dental prosthesis, according to one embodiment of the invention. The packaging device 1 is intended to protect a dental prosthesis between the manufacture of the latter and its use by a dentist. The packaging device 1 is a unitary packaging device, that is to say it is intended to enclose a single dental prosthesis. Alternatively, the packaging device could contain several dental prostheses. In particular, the dental prosthesis is a dental implant. The dental implant is intended to be screwed into a bone structure of the patient. On account of its integration into the bone of the patient, the dental implant requires a packaging device allowing optimum protection against any risk of contamination. Thus, the packaging device is intended to enclose the dental prosthesis hermetically or quasi-hermetically, in particular so as to prevent direct contact between the dental prosthesis and the fingers of a user. Alternatively, the dental prosthesis could be different from a dental implant, for example a dental abutment, a dental crown or a dental bridge.

The packaging device 1 comprises a base 2 comprising a support 3 intended to hold the dental prosthesis, an envelope 4 formed of a first part 5 and a second part 6, and two arms 7, 8. A first arm 7 extends between the base 2 and the first part 5 of the envelope 4. Similarly, a second arm 8 extends between the base 2 and the second part 6 of the envelope 4. The first arm 7 therefore comprises a first end 7A connected to the base 2, and a second end 7B, opposite the first end 7A, connected to the first part 5 of the envelope 4. Similarly, the second arm 8 comprises a first end 8A connected to the base 2, and a second end 8B, opposite the first end 8A, connected to the second part 6 of the envelope 4. The two parts 5 and 6 of the envelope are therefore connected to the base 2 by means of the two arms 7 and 8, respectively.

In FIG. 1, the packaging device is shown in its original configuration, that is to say in a state in which the packaging device is manufactured, without deformation. The packaging device is able to transition from its original configuration to a closed configuration (illustrated in FIG. 2). In the closed configuration, the first part 5 and the second part 6 of the envelope 4 are brought closer together so as to surround the dental prosthesis. In particular, the two parts 5 and 6 are in contact with each other along a line of contact. In the open configuration (illustrated in FIG. 8), the first part 5 and the second part 6 of the envelope 4 are spaced apart from each other, without contact between them, so as to provide access to the dental prosthesis fastened to the support 3. At a minimum, the packaging device 1 is able to transition once from its original configuration to its closed configuration (during packaging of the dental prosthesis), and then once from its closed configuration to its open configuration (during unpackaging of the dental prosthesis). The packaging device 1 can thus be for single use. Alternatively, the packaging device 1 could also be reused several times.

According to the embodiment shown, the envelope 4 has a generally cylindrical shape with a circular base when the packaging device is in the closed configuration. The first part 5 and the second part 6 of the envelope are two half-shells of the envelope and of substantially identical shape. In particular, they each comprise the shape of a half-cylinder. Alternatively, any other shape could be proposed, for example a cylindrical shape with a polygonal base, in particular a rectangular base, or a spherical or ovoid shape. An axis Z can be defined as being an axis parallel to a generatrix of the cylindrical shape of the envelope and passing through the center of the latter. The base 2 has a generally elongate shape along the axis Z. The first end 7A, 8A of each arm 7, 8 is connected to a first end 2A of the base 2. The support 3 is arranged at a second end 2B of the base, the second end 2B being opposite the first end 2A. In particular, the support 3 is in the form of a blind opening extending along the axis Z. This blind opening can have a profiled section allowing the insertion and removal of the dental prosthesis by a simple translational movement parallel to the axis Z. In the context of the description, it is assumed that the axis Z is a vertical axis and that the base rests on a horizontal plane. The terms “lower” and “upper” refer to an arrangement along the axis Z. The packaging device is advantageously symmetrical or quasi-symmetrical with respect to a plane passing through the axis Z.

The base 2 can also be inscribed in a cylindrical shape centered on the axis Z. Advantageously, the base 2 has a notched shape enabling each arm 7, 8 to bend in the direction of the base 2. The base 2 thus comprises a relatively thin central part 9 surrounded by a lower part 10 and a wider upper part 11. The base 2 thus has the overall shape of an hourglass.

The base 2 comprises two pivot means 12 and 13, respectively, for each of the two arms 7 and 8. The two pivot means 12, 13 are arranged at the second end 2B of the base, that is to say at its upper part 11. The two pivot means 12, 13 can be in the form of a plate extending substantially perpendicularly to the axis Z. When the packaging device is in a closed configuration, the second ends 7B and 8B of each arm may be in contact with or at a short distance from the pivot means 12 and 13, respectively. The pivot means 12, 13 are intended to form pivot points for each of the two arms 7 and 8. Bending of the first arm 7, between its first end 7A and the first pivot means 12, causes the first part 5 of the envelope 4 to pivot about the first pivot means 12. Similarly, bending of the second arm 8, between its first end 8A and the second pivot means 13, causes the second part 6 of the envelope 4 to pivot about the second pivot means 13.

The packaging device also comprises a retention means configured to maintain the packaging device in a closed configuration. This retention means comprises two tabs 16 for retaining the first part 5 of the envelope 4 in the closed configuration, and two tabs 17 for retaining the second part 6 of the envelope 4 in the closed configuration. The tabs 16 and 17 are intended to cooperate respectively with the second ends 7B and 8B of each arm. The tabs 16, 17 form fastening clips that are able to exert a retaining force on the back of each arm. The tabs 16 and 17 thus make it possible to hold the arms 7 and 8 respectively in abutment against the pivot means 12 and 13 or at a short distance from the pivot means 12 and 13. Alternatively, a single tab 16, 17 could be sufficient to retain the first part 5 or the second part 6 in the closed configuration. Each tab 16, 17 is intended to deform in order to allow the movement of the at least one arm when the packaging device transitions from its original configuration to its closed configuration.

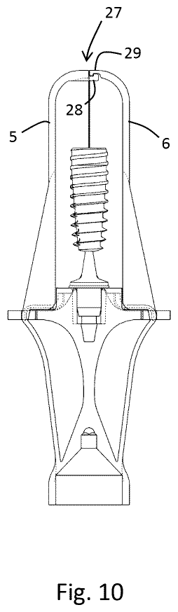

According to a variant embodiment illustrated in FIG. 10, the retention means described above can be replaced or supplemented by a second retention means. The second retention means is formed by a fastening clip 27 arranged between the two parts 5 and 6 of the envelope 4. The parts 5, 6 of the envelope 4 each comprise an interlocking surface 28, 29 cooperating together to maintain the packaging device in the closed configuration. These interlocking surfaces 28, 29 can extend along the entire line of contact between the first part 5 and the second part 6 of the envelope. In this case, they can advantageously form a baffle which improves the sealing of the packaging device. Alternatively, the interlocking surfaces can extend along only part of this line of contact, for example only at the upper end of the parts 5 and 6 of the envelope 4. Thus, the fastening clip is simple to produce, and the effort required to open the packaging device is reduced. According to another variant, the two parts 5 and 6 could also be weakly welded or glued to each other when the packaging device is in the closed configuration

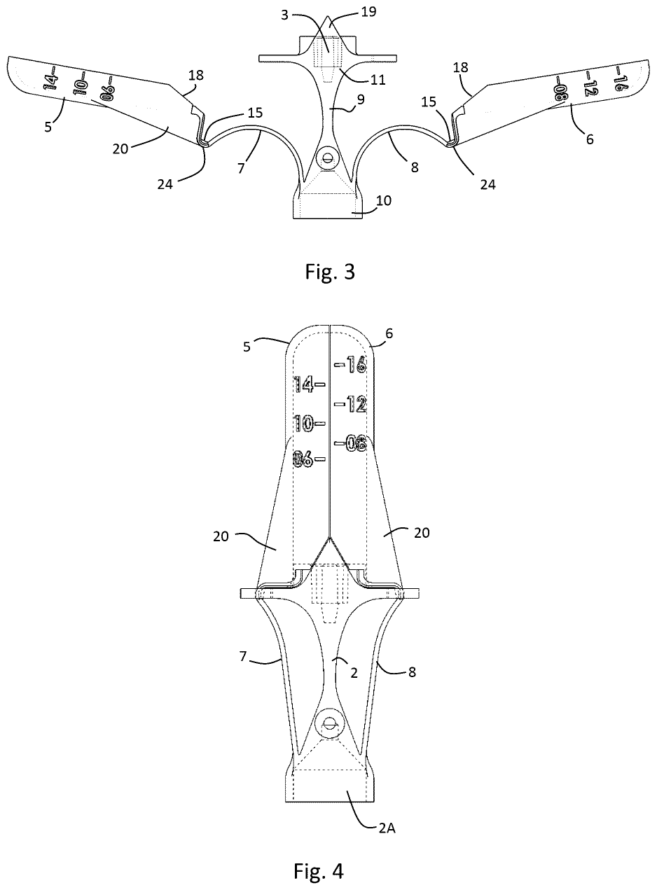

As is illustrated in FIG. 3, each of the two parts 5 and 6 of the envelope has recesses 18, or cutouts 18, at their lower end. These recesses 18 allow each of the two parts 5 and 6 to pivot between the original configuration or the open configuration and the closed configuration without interfering with the pivot means 12 and 13. The upper part 11 of the base 2 comprises two flanks 19 framing the support 3 and surmounting the plates in which the pivot means 12 and 13 are formed. The two flanks 19 have a shape adapted to fill the recesses 18 when the packaging device is in the closed configuration. In this case, the two flanks 19 have a generally triangular shape.

When the packaging device is in its original configuration, the two arms 7 and 8 have the shape of an arc of a circle. The two parts 5 and 6 of the envelope extend substantially horizontally and below the level of the dental prosthesis fastened to the support 3. The dental prosthesis is then very widely accessible. When the packaging device is in the closed configuration, the two arms 7 and 8 comprise a portion of rectilinear shape. This rectilinear shape extends from the first end 2A of the base to the pivot means 12, 13. Each arm 7, 8 also comprises a curvature 24 respectively around the pivot means 12, 13 at its second end 7B, 8B. The arms 7, 8 thus terminate in horizontal portions which extend from the pivot means 12, 13 to the lower end of each part 5, 6 of the envelope.

Advantageously, the packaging device comprises first ribs 20 extending over an outer face of each part 5, 6 of the envelope as far as its respective arm 7, 8. The packaging device also comprises second ribs 15 arranged inside the curvatures 24 of each arm 7, 8. The plate in which the two pivot means 12, 13 are formed advantageously comprises two notches 14 in which the second ribs 15 are positioned when the packaging device is in the closed configuration. The first ribs 20 and the second ribs 15 make it possible to stiffen the interface between each part 5, 6 of the envelope 4 and its respective arm 7, 8, so that a pivoting movement of the second end of each arm does indeed induce a pivoting movement of the corresponding envelope part. Alternatively, the packaging device could comprise only the first ribs 20 or only the second ribs 15, or any other form of rib, or even no rib, provided that the connection between each arm and the corresponding envelope part is sufficiently rigid, for example by virtue of sufficiently large material thicknesses.

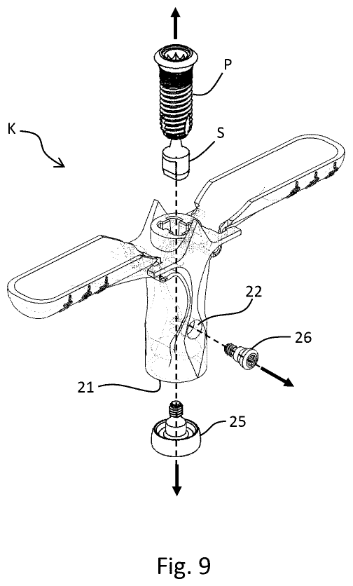

According to a refinement of the invention, the base 2 comprises at least one housing for a fastening screw able to cooperate with the dental prosthesis. Specifically, the base 2 comprises two housings 21, 22 in which two fastening screws 25, 26 of different sizes can be placed. The housings are blind openings of circular cross section. A first housing 21 extends in the lower part 10 of the base parallel to the axis Z. A second housing 22 also extends in the lower part 10 of the base, perpendicularly with respect to the axis Z, between the two arms 7 and 8. It is thus possible to provide a kit K, illustrated in FIG. 9, comprising the dental prosthesis P fastened to the support 3, and two fastening screws 25, 26 arranged in each of the two housings 21, 22. Alternatively, the kit could comprise only one of the two fastening screws or even any other accessory intended to cooperate with the dental prosthesis.

Advantageously, the packaging device is a one-piece element manufactured by plastic injection. It is therefore particularly simple to manufacture and inexpensive. By definition, the packaging device is manufactured in its original configuration, that is to say in the configuration of FIGS. 1, 3 and 5. On the one hand, this has the advantage that the packaging device is immediately ready to receive a dental prosthesis in its support. On the other hand, this gives the packaging device a shape memory corresponding to its original configuration. The arms 7 and 8 thus have an elasticity tending to move each part 5, 6 of the envelope toward a position corresponding to the closed configuration of the packaging device, after the first ends 7A, 8A of each arm are clipped into the fastening clips formed by the tabs 16 and 17. This allows the packaging device to be properly maintained in the closed configuration.

The first part 5 and/or the second part 6 of the envelope 4 can be made of transparent plastic material and can have graduations 23 in relief, which are configured for estimating a size of the dental prosthesis contained in the envelope 4. The graduations 23 can be formed in positive relief (that is to say projecting) or in negative relief (that is to say hollow). A user can thus compare the height of the dental prosthesis, held in the support 3, against the graduations 23 and estimate its size without having to open the envelope 4. The graduations 23 can be formed directly during the process of plastic injection of the packaging device. Thus, the integration of these graduations 23 does not require a specific marking operation.

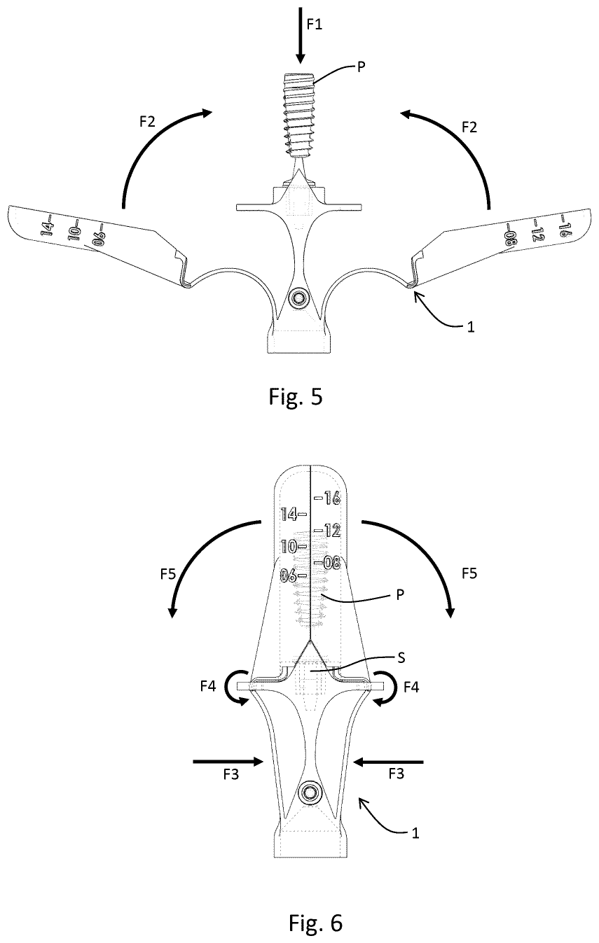

With reference to FIG. 5, in order to pack a dental prosthesis P in the packaging device 1, it is possible to proceed as follows. The packaging device is manufactured by plastic injection. The dental prosthesis P is manufactured in parallel. According to the embodiment illustrated in FIG. 5, the dental prosthesis P is a dental implant intended to be screwed into a bone structure of the patient. The dental implant is connected to a breakable part S (clearly visible in FIG. 9) by means of which it is fastened to the support 3. Alternatively, the dental implant might not be connected to such a breakable part and could instead be fastened directly to the support 3. However, the use of a breakable part has the advantage that the dental prosthesis P is kept out of contact with the packaging device 1, which avoids any risk of contamination. The breakable part S is inserted into the support 3 by a simple translational movement parallel to the axis Z, as is indicated by the arrow F1. The first part 5 and the second part 6 of the envelope are then closed around the dental prosthesis P. The two parts 5 and 6 pivot about a quarter of a turn in the direction of the axis Z, as is indicated by the arrows F2. When the packaging device is about to reach its closed configuration, the tabs 16 and 17 deform slightly in order to allow the arms 7 and 8 to pass against the pivot means 12 and 13. If necessary, the interlocking surfaces 28, 29 engage one against the other. The packaging device is thus properly maintained in the closed configuration. A kit K is thus obtained comprising the packaging device 1 and the dental prosthesis P fastened to the support 3 of the packaging device. The kit K can be completed by fastening a screw 25 or 26 or the two screws 25 and 26 in the housings 21 and 22 provided for this purpose. The dental prosthesis is thus well protected and can be transported from the site of its manufacture to the site of its use and stored without risk of contamination.

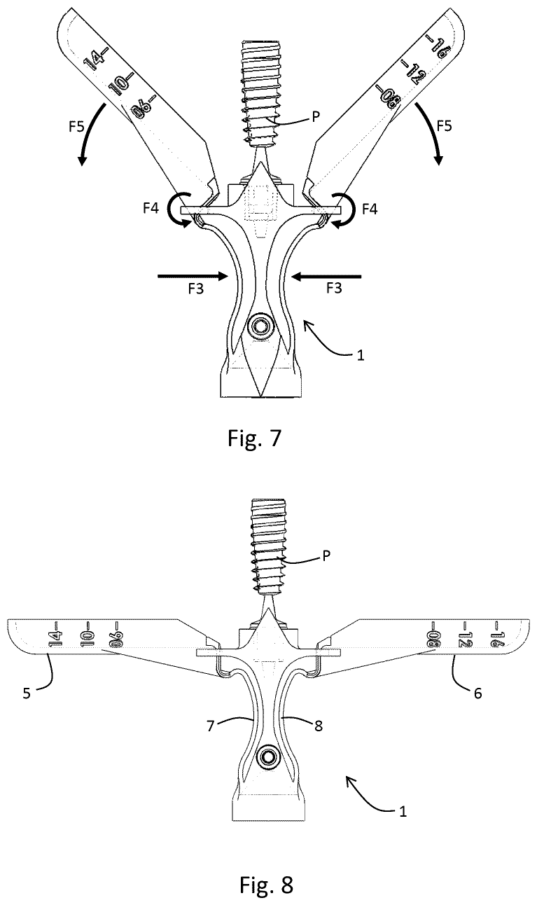

Then, when a dentist wishes to fit a dental prosthesis in the mouth of the patient, he can first of all easily select the correct size of dental prosthesis by virtue of the graduations 23. The packaging device can then advantageously be manipulated with one hand. The first arm 7 and the second arm 8 can be pressed or pinched against each other by means of the thumb and index finger of one hand, substantially between their two ends, as is indicated by the arrows F3. This causes the two arms 7 and 8 to bend in the direction of the base. The two arms 7, 8 thus transition from a substantially rectilinear shape to a shape curved in the direction of the base, as can be seen in FIGS. 7 and 8. The bending of the two arms 7 and 8 causes a tensile force on the second ends 7B and 8B, respectively By a reaction force of the pivot means 12, 13, this traction force results in a rotation of the second ends 7B and 8B about the pivot means 12, 13, as is indicated by the arrows F4. As the two parts 5 and 6 of the envelope are connected to the second ends 7B and 8B, they likewise pivot in accordance with the arrows F5. The ribs 15, 20 make it possible to stiffen the connection between the second ends 7B and 8B and the two parts 5 and 6 of the envelope, which makes it possible to transfer the force exerted on the arms 7 and 8 to the two parts 5 and 6 of the envelope. Thus, the two parts 5 and 6 of the envelope move apart, and the dentist is able to access the dental prosthesis. The two arms 7, 8 are flexible insofar as they can be sufficiently bent by a pinching force between two fingers so as to induce a pivoting movement of the two parts of the envelope.

Since opening the packaging device requires only the use of one hand, the dentist is able to easily grasp the dental prosthesis P with the other hand without any risk of dropping it. If necessary, he can exert a torsional force on the dental prosthesis P so as to detach it from the breakable part S, which then remains in place in the support 3.

In the open configuration, the two arms 7, 8 remain held by the retention means. The two parts 5 and 6 of the envelope can extend along two axes forming an angle of about 180°, as can be seen in FIG. 8. Alternatively, the open configuration of the packaging device might not correspond exactly to the configuration illustrated in FIG. 8. On the contrary, in the open configuration, the packaging device may be in an intermediate configuration between the closed configuration (illustrated in FIGS. 2, 4 and 6) and the configuration of FIG. 8. For example, the open configuration could also match the configuration in FIG. 7. In the open configuration, the two parts 5 and 6 of the envelope can extend along two axes preferably forming an angle greater than or equal to 60°, in particular greater than or equal to 90°. In all cases, in the open configuration, the two parts 5 and 6 of the envelope are sufficiently spaced apart to allow easy access to the dental prosthesis.

According to a variant embodiment (not shown), the packaging device could comprise only one of the two arms 7, 8, as described above. Only one of the two parts 5, 6 of the envelope 4 could then be fixed relative to the base 2. The movement of the part 5, 6 connected to the single arm would suffice to give access to the dental prosthesis. In such a variant embodiment, the two parts 5 and 6 of the envelope could advantageously be asymmetrical. Such a variant of the packaging device could therefore be even simpler to manufacture. However, a packaging device in which the two parts of the envelope are movable relative to the base, and which comprises two arms, each connected to one part of the envelope, has the advantage of offering a greater spacing between the two parts of the envelope when it is in the open configuration, which facilitates access to the dental prosthesis.

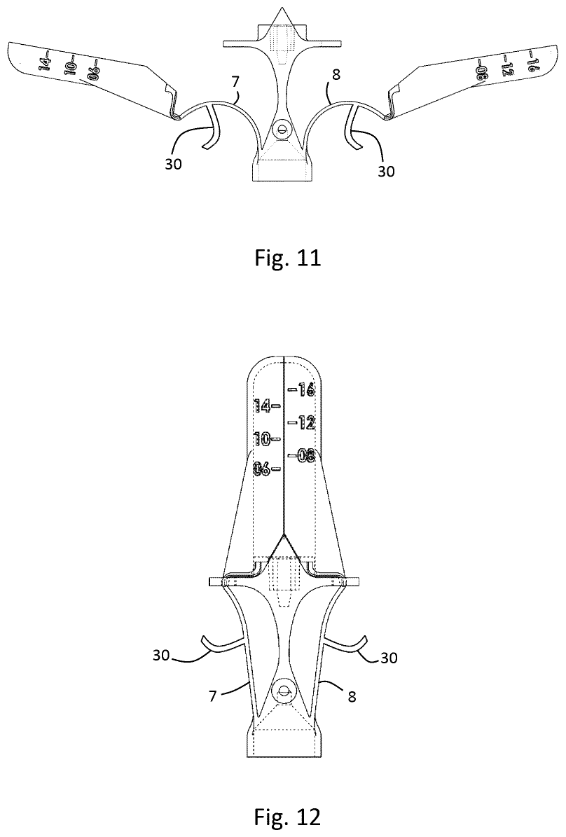

According to another variant embodiment, shown in FIGS. 11 and 12, each arm 7, 8 could comprise a bearing surface 30 extending substantially radially outward, that is to say perpendicularly with respect to the axis Z. To open the packaging device, the user could position his thumb on a lower face of the base 2 and his index finger and middle finger, respectively, on the two bearing surfaces. The bearing surfaces could possibly have a curved shape adapted to receive the index finger and the middle finger. This gripping is similar to the gripping of a syringe. To open the packaging device, the user could exert a downward traction force on each bearing surface (in the same way as a syringe is pressed). This traction force would induce the pivoting movement of the two parts of the envelope by reaction of the second ends on the pivot means, as is described above.

The various embodiments described can be combined with one another. In particular, the variant embodiment of FIGS. 11 and 12 can be made with a single arm from the two arms 7 and 8. The variant embodiment of FIGS. 11 and 12 and also the variant embodiment with a single arm from the two arms 7 and 8 can comprise a fastening clip 27 arranged between the two parts of the envelope.

1. A packaging device for a dental prosthesis, comprising:
a base comprising a support intended to hold the dental prosthesis,
an envelope formed of a first part and of a second part, and
at least one arm comprising a first end and a second end, the first end being connected to the base, the second end being connected to the first part of the envelope,
the packaging device being able to transition from a closed configuration to an open configuration, the first part and the second part of the envelope being brought close together to enclose the dental prosthesis when the packaging device is in the closed configuration, the first part and the second part of the envelope being spaced apart from each other to provide access to the dental prosthesis when the packaging device is in the open configuration, and
the base comprising at least one pivot means for the at least one arm, a force applied to the at least one arm, between the first end of the at least one arm and the at least one pivot means, causing the packaging device to transition from the closed configuration to the open configuration.

2. The packaging device as claimed in claim 1, wherein
the packaging device comprises a first arm and a second arm, each arm comprising a first end and a second end, the first end of each arm being connected to the base, the second end of the first arm being connected to the first part of the envelope, the second end of the second arm being connected to the second part of the envelope,
the base comprising a first pivot means for the first arm and a second pivot means for the second arm,
a force applied to the first arm, between its first end and the first pivot means, and a force applied to the second arm, between the first end of the second arm and the second pivot means, causing the packaging device to transition from the closed configuration to its open configuration.

3. The packaging device as claimed in claim 1, wherein the packaging device is in one piece and obtained by plastic injection.

4. The packaging device as claimed in claim 1, wherein the base comprises at least one retention means cooperating with the at least one arm in order to hold the packaging device in the closed configuration.

5. The packaging device as claimed in claim 1, wherein the retention means comprises at least one deformable tab, the tab being intended to deform in order to allow movement of the at least one arm when the packaging device transitions from an original configuration at the end of manufacture to the closed configuration.

6. The packaging device as claimed in claim 1, wherein the packaging device comprises a shape memory that tends to cause the packaging device to transition to the closed configuration.

7. The packaging device as claimed in claim 1, wherein the base comprises a generally elongate shape, the first end of the at least one arm being connected to a first end of the base, the support being arranged at a second end of the base, the second end of the base being opposite the first end of the base.

8. The packaging device as claimed in claim 1, wherein:
the base further comprises at least one housing for a fastening screw able to cooperate with the dental prosthesis, and/or
the first part and the second part of the envelope are two half-shells of the envelope, and/or
the first part and/or the second part of the envelope are made of transparent plastic material and have graduations in relief, which are configured for estimating a size of the dental prosthesis.

9. The packaging device as claimed in claim 1, wherein the packaging device comprises a fastening clip arranged between the first part and the second part of the envelope, wherein the fastening clip is adapted to maintain the packaging device in the closed configuration.

10. A kit comprising
the packaging device as claimed in claim 1, and
a dental prosthesis fastened to the support of the base.

11. A method for manufacturing a packaging device, comprising:
manufacturing the packaging device as claimed in claim 1 by plastic injection in an original configuration,
wherein, in the original configuration, the at least one arm has a shape of an arc of a circle and the at least one arm is spaced apart from the at least one pivot means.

12. A method for packaging a dental prosthesis in a packaging device, comprising:
manufacturing the packaging device as claimed in claim 1 by plastic injection, then
fastening a dental prosthesis to the support of the base, then
bringing the first part of the envelope and the second part of the envelope together around the dental prosthesis.

13. A method for unpackaging a dental prosthesis packaged in the packaging device as claimed in claim 1, comprising:
pressing against the at least one arm, between the first end of the at least one arm and the at least one pivot means, so as to cause the at least one arm to bend in the direction of the base,
wherein the bending of the at least one arm causes the packaging device to open.

14. The packaging device as claimed in claim 2, wherein the packaging device is in one piece and obtained by plastic injection.

15. The packaging device as claimed in claim 14, wherein the base comprises at least one retention means cooperating with the at least one arm in order to hold the packaging device in the closed configuration.

16. The packaging device as claimed in claim 2, wherein the base comprises at least one retention means cooperating with the at least one arm in order to hold the packaging device in the closed configuration.

17. The packaging device as claimed in claim 3, wherein the base comprises at least one retention means cooperating with the at least one arm in order to hold the packaging device in the closed configuration.

18. The packaging device as claimed in claim 2, wherein the retention means comprises at least one deformable tab, the tab being intended to deform in order to allow movement of the at least one arm when the packaging device transitions from an original configuration at the end of manufacture to the closed configuration.

19. The packaging device as claimed in claim 3, wherein the retention means comprises at least one deformable tab, the tab being intended to deform in order to allow movement of the at least one arm when the packaging device transitions from an original configuration at the end of manufacture to the closed configuration.

20. The packaging device as claimed in claim 4, wherein the retention means comprises at least one deformable tab, the tab being intended to deform in order to allow movement of the at least one arm when the packaging device transitions from an original configuration at the end of manufacture to the closed configuration.
